(19) United States
(12) Patent Application Publication
(10) Pub. No.: US 2013/0032786 A1
(43) Pub. Date: Feb. 7, 2013

(54) ORGANIC COMPOUND AND ORGANIC ELECTROLUMINESCENT DEVICE EMPLOYING THE SAME

(71) Applicants: Heh-Lung HUANG, New Taipei City (TW); Cheng-An Wu, New Taipei City (TW); Chien-Hong Cheng, Hsin-Chu City (TW)

(72) Inventors: Heh-Lung HUANG, New Taipei City (TW); Cheng-An Wu, New Taipei City (TW); Chien-Hong Cheng, Hsin-Chu City (TW)

(21) Appl. No.: 13/243,552

(22) Filed: Sep. 23, 2011

(30) Foreign Application Priority Data
Aug. 5, 2011 (TW) 100127919

(51) Int. Cl. H01L 51/54; C07D 403/14; C07D 403/10

(52) U.S. Cl. 257/40; 548/266.4; 257/E51.024

(57) ABSTRACT

Organic compounds and organic electroluminescence devices employing the same are provided. The organic compound has a chemical structure represented below:

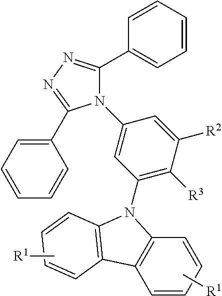

wherein, R¹ are independent and can be hydrogen, or C₁₋₈ alkyl, and R², and R³ can be hydrogen, hydroxy, C₁₋₈ alkyl, C₁₋₈ alkoxy, C₅₋₁₀ aryl, or C₂₋₈ heteroaryl. The organic compounds of the disclosure have a high triplet energy (tEg) gap and apt to transmit the energy to a guest emitter.

### CROSS REFERENCE TO RELATED APPLICATIONS

This application is based upon and claims the benefit of priority from the prior Taiwan Patent Application No. 100127919, filed on Aug. 5, 2011, the entire contents of which are incorporated herein by reference.

### BACKGROUND

1. Field

The disclosure relates to an organic compound and organic electroluminescence device employing the same and, more particularly, to an organic compound serving as a host material and a phosphorescent organic electroluminescence device employing the same.

2. Description of the Related Art

Recently, with the development and wide application of electronic products, such as mobile phones, PDAs, and notebook computers, there has been increasing demand for flat display elements which consume less electric power and occupy less space. Organic electroluminescent devices are self-emitting and highly luminous, with wider viewing angles, faster response speeds, and simpler fabrication methods, making them an industry display of choice.

Generally, an organic electroluminescent device is composed of a light-emission layer sandwiched between a pair of electrodes. When an electric field is applied to the electrodes, the cathode injects electrons into the light-emission layer and the anode injects holes into the light-emission layer. When the electrons recombine with the holes in the light-emission layer, excitons are formed. Recombination of the electron and hole results in light emission.

Depending on the spin states of the hole and electron, the exciton which results from the hole and electron recombination can have either a triplet or singlet spin state. Luminescence from a singlet exciton results in fluorescence whereas luminescence from a triplet exciton results in phosphorescence. The emissive efficiency of phosphorescence is three times that of fluorescence. Therefore, it is crucial to develop highly efficient phosphorescent material, in order to increase the emissive efficiency of the OLED.

In application of organic electroluminescent devices, phosphorescent guest materials have to be used in combination with host materials which has an energy gap matched therewith, thereby achieving optimal electroluminescent performance and quantum yield. Particularly, since blue and green host materials require larger differences of energy gap between the host and guest material for electroluminescence, the host materials used in an phosphorescent OLED should have a shorter conjugated system. Further, in order to keep the key characteristics of the organic compound used in OLEDs (i.e. thermal-stability), the host material should also have larger molecular weight, resulting in difficulties for chemical structure designs.

Since conventional, commercially available phosphorescent host materials or phosphorescent host materials disclosed in prior art references merely have the moieties of carbazole or silyl benzene derivatives, the phosphorescent host materials exhibit inferior thermal stability, resulting in devices made therefrom to have low current density and high operating voltage.

### BRIEF SUMMARY

An exemplary embodiment of an organic compound has a Formula (I), of:

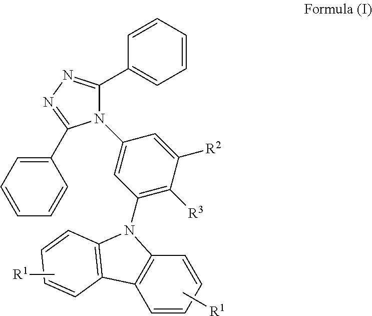

wherein, R¹ are independent a hydrogen, or C₁₋₈ alkyl; and R², and R³ can be hydrogen, hydroxy, C₁₋₈ alkyl, C₁₋₈ alkoxy, C_{5.10} aryl, or C₂₋₈ heteroaryl.

In another exemplary embodiment of the disclosure, an organic electroluminescence device is provided. The device includes: a pair of electrodes; and an electroluminescent element disposed between the pair of electrodes, wherein the electroluminescent element includes the aforementioned organic compound.

Yet another exemplary embodiment of the disclosure provides an organic electroluminescence device including an emission layer which includes a host material and a phosphorescent dopant. Particularly, the host material includes the aforementioned organic compound and the emission layer emits blue or green light under a bias voltage.

A detailed description is given in the following embodiments with reference to the accompanying drawings.

### BRIEF DESCRIPTION OF THE DRAWINGS

The disclosure can be more fully understood by reading the subsequent detailed description and examples with references made to the accompanying drawings, wherein:

FIG. 1 shows a cross section of an organic electroluminescent device disclosed by an embodiment of the disclosure.

### DETAILED DESCRIPTION

The following description is of the best-contemplated mode of carrying out the disclosure. This description is made for the purpose of illustrating the general principles of the invention and should not be taken in a limiting sense. The scope of the invention is best determined by reference to the appended claims.

Organic Compound

The disclosure provides organic compounds having a high triplet energy (tEg) gap and apt to transmit the energy to a guest emitter. Therefore, the organic compounds of the disclosure are suitable as host material of blue or green phosphorescent organic electroluminescent devices, thereby increasing the efficiency thereof.

The disclosure provides an organic compound having a Formula (I), of:

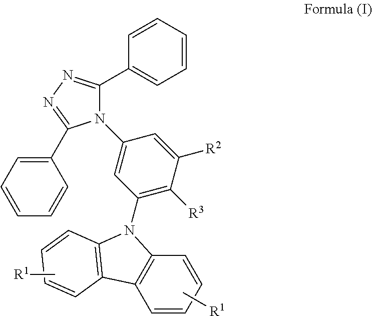

wherein, R¹ are independent and can be hydrogen, or C₁₋₈ alkyl; and R², and R³ can be hydrogen, hydroxy, C₁₋₈ alkyl, C₁₋₈ alkoxy, C₅₋₁₀ aryl, or C₂₋₈ heteroaryl.

In the structure of Formula (I), the moieties

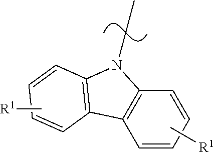

means that R¹ can be located at any one of the four substitutable positions of the benzene ring and the R¹ are independent. For example, R¹ can be each independently a methyl group, ethyl group, propyl group, isopropyl group, butyl group, tert-butyl group, pentyl group, or hexyl group. Further, R² and R³ can be each independently methyl group, ethyl group, propyl group, isopropyl group, butyl group, tert-butyl group, pentyl group, hexyl group, methoxy group, ethoxy group, propoxy group, isopropoxy group, butoxy group, isobutoxy group, phenyl, biphenyl, pyridyl, furyl, naphthyl, anthryl, phenanthrenyl, imidazolyl, pyrimidinyl, quinolinyl, indolyl, or thiazolyl.

According to some embodiments of the disclosure, the organic compound of the disclosure can have a structure represented by Formula (II) or Formula (III), of:

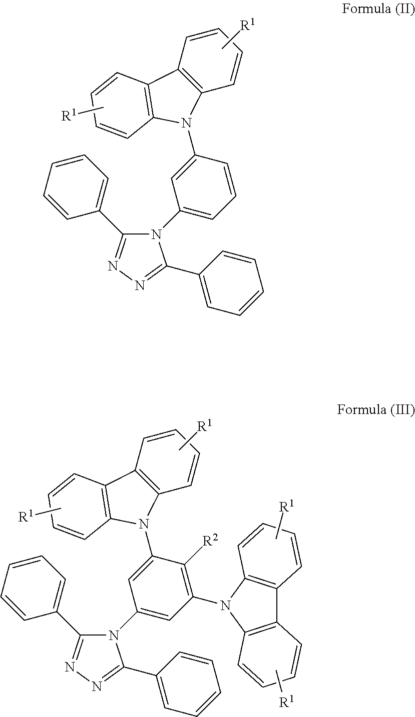

wherein, R¹ are independent and can be hydrogen, or C₁₋₈ alkyl; and R² can be hydrogen, hydroxy, C₁₋₈ alkyl, or C₁₋₈ alkoxy.

The organic compounds of Formula (I) of the disclosure have a high triplet energy (tEg) gap and are apt to transmit the energy to a guest emitter. Further, in comparison with the conventional compound (represented by

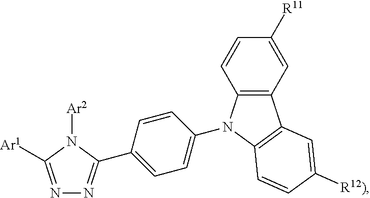

wherein Ar¹ and Ar² are phenyl or pyridine group, and R¹¹ and R¹² are hydrogen, alkyl group, hydroxyl group, or aryl group), since the organic compounds of Formula (I) of the disclosure have a trizole moiety bound to the benzene at the meta-position relative to a carbazole moiety resulting in a shorter conjugated system, the organic compounds of the disclosure are suitable for serving as a blue or green host material for a phosphorescent organic electroluminescent device. Moreover, since the organic compounds of Formula (I) of the disclosure, in comparison with the conventional compound, has a lower structural symmetry, the organic compound is not apt to be crystallized after formation of a film by evaporation.

The organic compounds according to Formula (I) of the invention include the following compounds shown in Table 1. In addition, the contraction thereof are also named and shown in Table 1.

TABLE 1

| Example | structure | contraction |
|---|---|---|
| 1 | 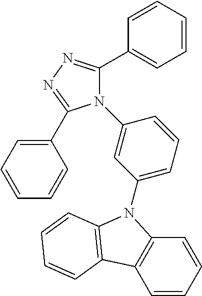 [SMILES: c1ccc(-c2nnc(-c3ccccc3)n2-c2cccc(-n3c4ccccc4c4ccccc43)c2)cc1] | m-TAZCz |
| 2 | 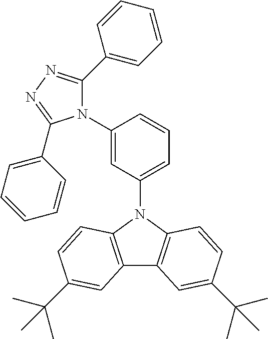 [SMILES: CC(C)(C)c1ccc2c(c1)c1cc(C(C)(C)C)ccc1n2-c1cccc(-n2c(-c3ccccc3)nnc2-c2ccccc2)c1] | m-TAZtCz |
| 3 | 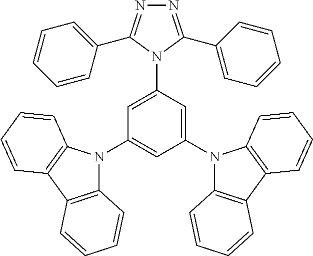 [SMILES: c1ccc(-c2nnc(-c3ccccc3)n2-c2cc(-n3c4ccccc4c4ccccc43)cc(-n3c4ccccc4c4ccccc43)c2)cc1] | m -TAZDCz |
| 4 | 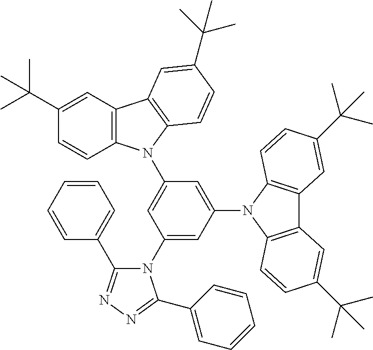 [SMILES: CC(C)(C)c1ccc2c(c1)c1cc(C(C)(C)C)ccc1n2-c1cc(-n2c(-c3ccccc3)nnc2-c2ccccc2)cc(-n2c3ccc(C(C)(C)C)cc3c3cc(C(C)(C)C)ccc32)c1] | m-TAZDtCz |
| 5 | 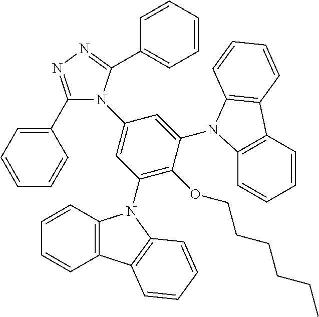 [SMILES: CCCCCCOc1c(-n2c3ccccc3c3ccccc32)cc(-n2c(-c3ccccc3)nnc2-c2ccccc2)cc1-n1c2ccccc2c2ccccc21] | m-TAZDCz-nH |

In order to clearly illustrate the method for preparing organic compounds according to Formula (I), the preparation of compounds disclosed in Examples 1-5 are described in detail as below.

### Example 1

### Preparation of Compound m-TAZCz

First, compound (1) (benzoyl chloride, 71.42 mmole, 10 g) was added into a 250 ml bottle and dissolved into THF (100 ml). Next, N₂H₄ (32.46 mmol, 1.62 g) was injected into the bottle at 0° C. After reacting for 4 hrs, a compound (2) was obtained with a yield of 92%. The synthesis pathway was as follows:

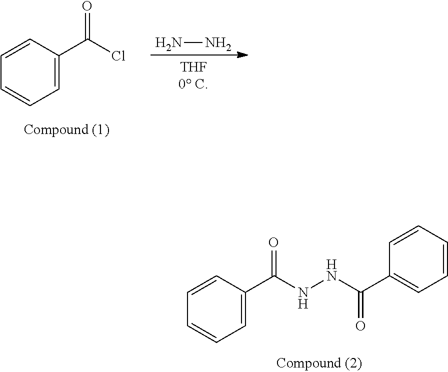

Next, compound (2) (41.66 mmol. 10 g), and PCL5 (91.51 mmol. 18.76 g) were added into a 250 ml bottle and dissolved into toluene (100 mL). Next, after heating to 120° C. for 3 hrs, the result was purified by column chromatography, obtaining compound (3) with a yield of 90%. The synthesis pathway was as follows:

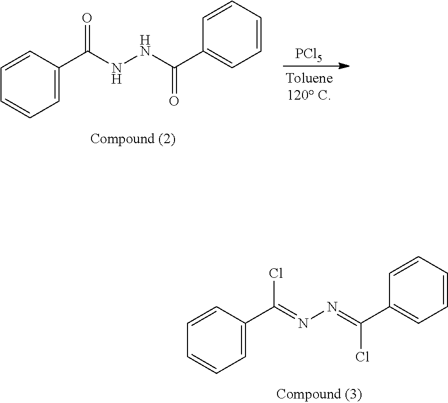

Next, compound (3) (18.11 mmol. 5 g), 3-bromoaniline (21.73 mmol, 3.69 g), and N,N-dimethyl aniline (25 mL) were added into a 100 ml bottle. Next, after heating to 130° C. for 12 hrs, the result was purified by column chromatography, obtaining compound (4) with a yield of 50%. The synthesis pathway was as follows:

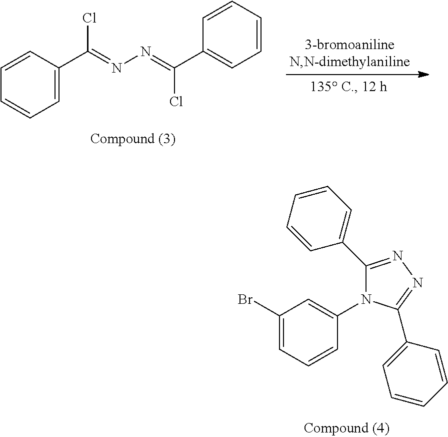 [SMILES: Cl/C(=N\N=C(/Cl)c1ccccc1)c1ccccc1 ; "3-bromoanilineN,N-dimethylaniline" ; Brc1cccc(-n2c(-c3ccccc3)nnc2-c2ccccc2)c1]

Next, compound (4) (8 mmol, 3 g), carbazole (9.6 mmol, 1.61 g) and K2CO3 (40 mmol, 5.52 g) were added into a 50 ml bottle and dissolved into DMSO (30 ml). The mixture was heated to 180° C. for 36 hrs. After cooling, the result was purified by washing with water, and compound m-TAZCz was obtained. The synthesis pathway was as follows:

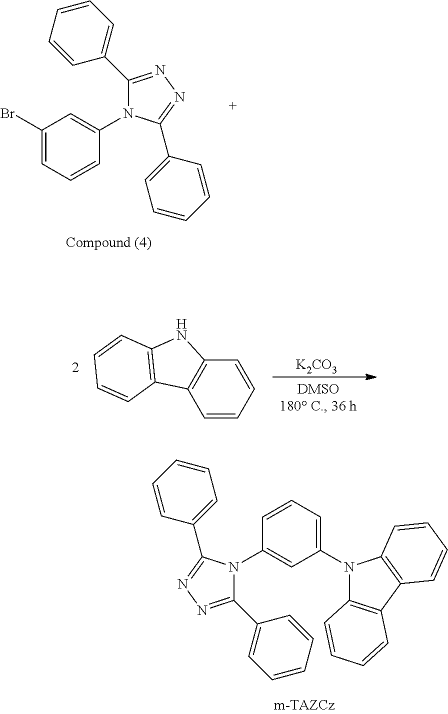

The physical measurements of the compound m-TAZCz are listed below:

1H-NMR (400 MHz, CDCl3, δ): 8.07 (m, 4H), 7.72-7.21 (m, 20H), 6.85 (m, 4H).

13C-NMR (100 MHz, CDCl3, δ): 154.70, 139.98, 139.38, 136.38, 131.22, 129.92, 129.17, 128.77, 127.42, 126.73, 126.15, 125.93, 123.68, 120.60, 120.44, 109.10.

HRMS (EI) Calcd for C32H22N4 (M+): 462.1844. Found: 462. 1844.

Elemental analysis: C, 83.09; H, 4.79; N, 12.11. Found: C, 83.33; H, 4.69; N, 12.22.

### Example 2

### Preparation of Compound m-TAZtCz

Compound (4) (13.33 mmol, 5 g), compound (5)

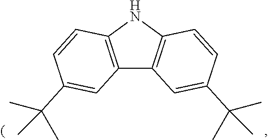 [SMILES: CC(C)(C)c1ccc2[nH]c3ccc(C(C)(C)C)cc3c2c1]

16 mmol, 4.46 g) and K2CO3 (66.66 mmol, 9.2 g) were added into a 50 ml bottle and dissolved into DMSO (30 ml). The mixture was heated to 180° C. for 36 hrs. After cooling, the result was purified by washing with water, and compound m-TAZtCz was obtained. The synthesis pathway was as follows:

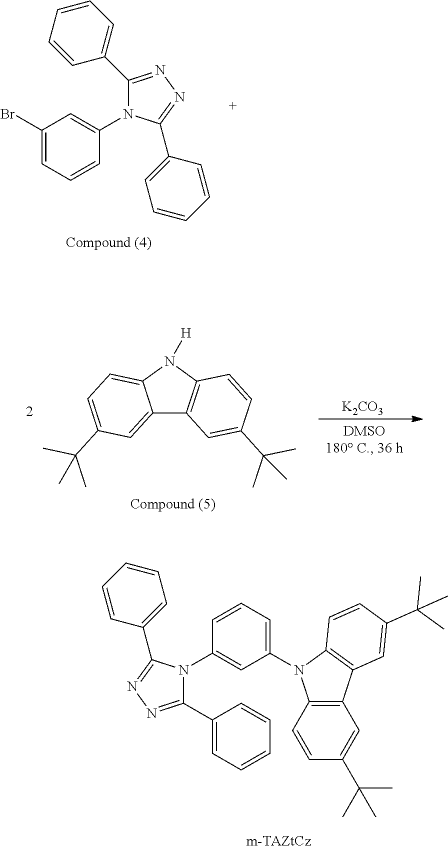 [SMILES: Brc1cccc(-n2c(-c3ccccc3)nnc2-c2ccccc2)c1 ; CC(C)(C)c1ccc2[nH]c3ccc(C(C)(C)C)cc3c2c1 ; "K2CO3" ; CC(C)(C)c1ccc2c(c1)c1cc(C(C)(C)C)ccc1n2-c1cccc(-n2c(-c3ccccc3)nnc2-c2ccccc2)c1]

The physical measurements of the compound m-TAZtCz are listed below:

1H-NMR (400 MHz, CDCl3, δ): 8.07 (s, 2H), 7.71-7.16 (m, 16H), 6.84 (d, J=8, 2H), 1.44 (s, 18H).

13C-NMR (100 MHz, CDCl3, δ): 143.69, 138.28, 131.21, 130.06, 129.12, 128.80, 125.38, 123.76, 116.43, 108.58, 34.69, 31.89.

HRMS (EI) Calcd for C32H22N4 (M+): 462.1844. Found: 462.1844.

HRMS (EI) Calcd for C40H38N4 (M+): 574.3096. Found: 574.3100. Elemental analysis: C, 83.59; H, 6.66; N, 9.75. Found: C, 83.69; H, 6.74; N, 9.78.

### Example 3

### Preparation of Compound m-TAZDCz

First, compound (3) (36.22 mmol, 10 g), compound (6)

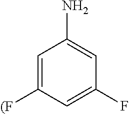

39.85 mmol, 5.14 g), and N,N-dimethyl aniline (30 mL) were added into a 250 ml bottle. Next, the mixture was heated to 135° C. for 12 hrs. After reaction, a compound (7) was obtained with a yield of 50%. The synthesis pathway was as follows:

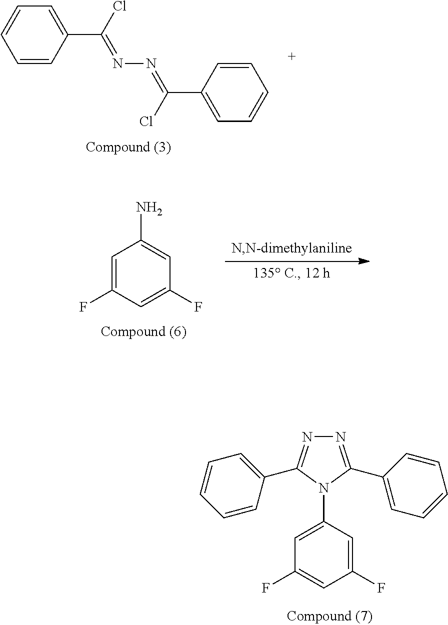 [SMILES: Cl/C(=N\N=C(/Cl)c1ccccc1)c1ccccc1 ; Nc1cc(F)cc(F)c1 ; Fc1cc(F)cc(-n2c(-c3ccccc3)nnc2-c2ccccc2)c1]

Next, compound (7) (15.01 mmol, 5 g), carbazole (33.03 mmol, 5.54 g) and K2CO3 (75.05 mmol, 10.35 g) were added into a 50 ml bottle and dissolved into DMSO (30 ml). The mixture was heated to 180° C. for 36 hrs. After cooling, the result was purified by washing with water, and compound m-TAZDCz was obtained with a yield of 82%. The synthesis pathway was as follows:

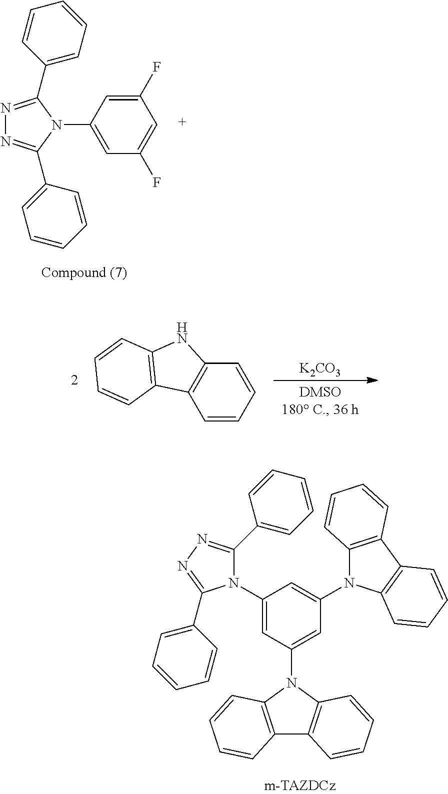

The physical measurements of the compound m-TAZDCz are listed below:

1H-NMR (400 MHz, CDCl3, δ): 8.07 (d, J=8 Hz, 4H), 7.97 (s, 1H), 7.68-7.55 (m, 11H), 7.34 (s, 2H), 7.29-7.23 (m, 7H), 6.92 (m, 4H).

13C-NMR (100 MHz, CDCl3, δ): 154.55, 140.64, 139.66, 130.17, 129.57, 129.15, 126.59, 126.35, 124.00, 123.87, 123.56, 120.99, 120.57, 109.14.

HRMS (EI) Calcd for C44H29N5 (M+): 627.2523. Found: 627.2428. Elemental analysis: C, 84.19; H, 4.66; N, 11.16. Found: C, 84.06; H, 4.69; N, 11.15.

### Example 4

### Preparation of Compound m-TAZDtCz

Compound (7) (15.01 mmol, 5 g), compound (5)

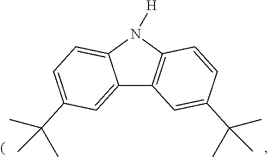 [SMILES: CC(C)(C)c1ccc2[nH]c3ccc(C(C)(C)C)cc3c2c1]

33.03 mmol, 9.22 g), and K2CO3 (75.07 mmol, 10.36 g) were added into a 50 ml bottle and dissolved into DMSO (30 ml). The mixture was heated to 180° C. for 36 hrs. After cooling, the result was purified by washing with water, and compound m-TAZDtCz. The synthesis pathway was as follows:

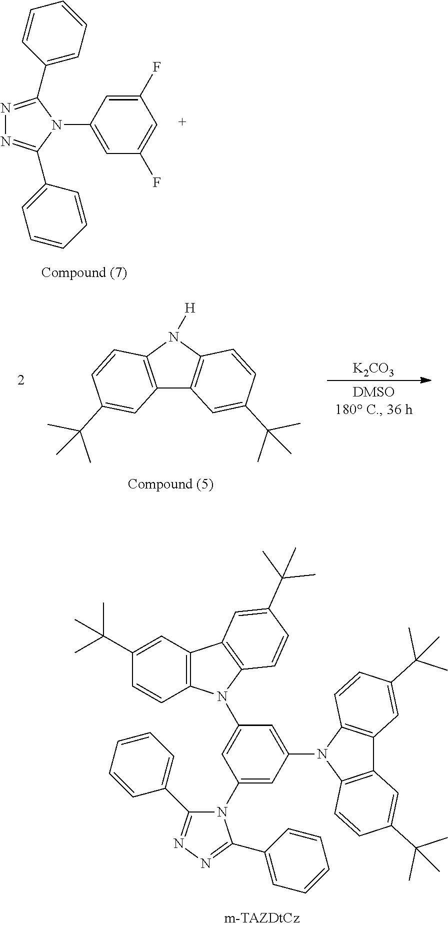 [SMILES: Fc1cc(F)cc(-n2c(-c3ccccc3)nnc2-c2ccccc2)c1 ; CC(C)(C)c1ccc2[nH]c3ccc(C(C)(C)C)cc3c2c1 ; "K2CO3" ; CC(C)(C)c1ccc2c(c1)c1cc(C(C)(C)C)ccc1n2-c1cc(-n2c(-c3ccccc3)nnc2-c2ccccc2)cc(-n2c3ccc(C(C)(C)C)cc3c3cc(C(C)(C)C)ccc32)c1]

The physical measurements of the compound m-TAZDtCz are listed below:

1H-NMR (400 MHz, CDCl3, δ): 8.10 (s, 4H), 8.09 (s, 1H), 7.66-7.28 (m, 16H), 6.89 (m, 4H), 1.47 (s, 36H).

13C-NMR (100 MHz, CDCl3, δ): 154.71, 144.04, 141.07, 138.02, 137.21, 130.03, 129.49, 129.08, 126.88, 123.97, 123.88, 123.14, 122.58, 116.55, 108.68, 34.71, 31.88.

Elemental analysis: C, 84.57; H, 7.22; N, 8.22. Found: C, 84.70; H, 7.60; N, 8.29.

### Example 5

### Preparation of Compound m-TAZDCz-nH

Compound (3) (36.23 mmol, 10 g), compound (8)

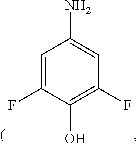

43.47 mmol, 6.30 g), and N,N-dimethyl aniline were added into a 100 ml reaction bottle. Next, the mixture was heated to 135° C. for 12 hrs. After reaction, a compound (9) was obtained with a yield of 50%. The synthesis pathway was as follows:

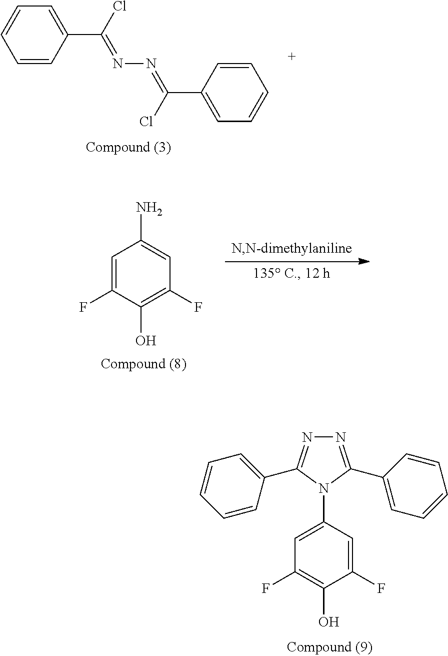 [SMILES: Cl/C(=N\N=C(/Cl)c1ccccc1)c1ccccc1 ; Nc1cc(F)c(O)c(F)c1 ; Oc1c(F)cc(-n2c(-c3ccccc3)nnc2-c2ccccc2)cc1F]

Compound (9) (14.32 mmol, 5 g), carbazole (31.51 mmol, 5.29 g), and K2CO3 (71.6 mmol. 9.8 g) were added into a 50 ml bottle and dissolved into DMSO (30 ml). The mixture was heated to 180° C. for 36 hrs. After cooling, the result was purified by washing with water, and compound (10) was obtained with a yield of 90%. The synthesis pathway was as follows:

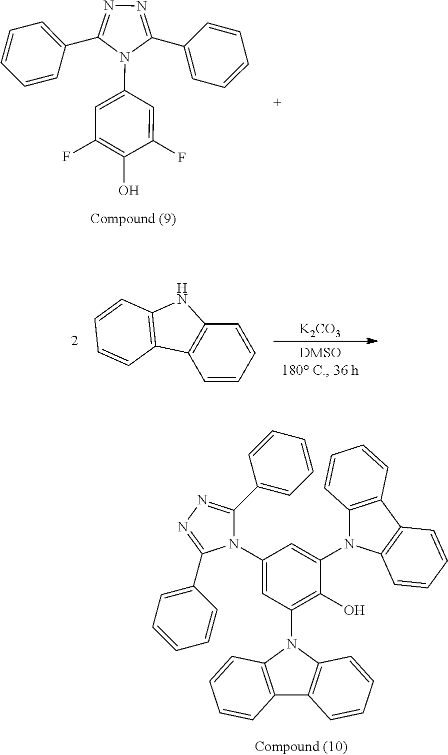

Next, compound (X) (3.1 mmol, 2 g), 1-Bromohexane (3.73 mmol, 0.61 g), and KOH (4.04 mmol, 0.22 g) were added into a 100 ml bottle and dissolved into ethanol (30 ml). The mixture was heated to reflux for 3 hrs. After purification, compound m-TAZDCz-nH was obtained with a yield of 98%. The synthesis pathway was as follows:

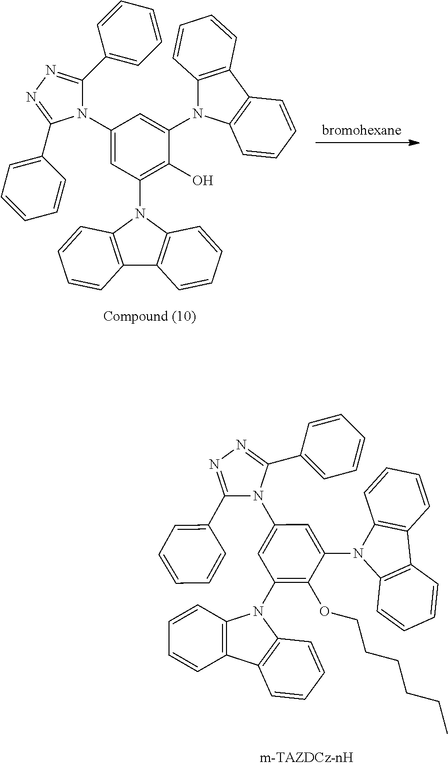 [SMILES: Oc1c(-n2c3ccccc3c3ccccc32)cc(-n2c(-c3ccccc3)nnc2-c2ccccc2)cc1-n1c2ccccc2c2ccccc21 ; CCCCCCOc1c(-n2c3ccccc3c3ccccc32)cc(-n2c(-c3ccccc3)nnc2-c2ccccc2)cc1-n1c2ccccc2c2ccccc21]

The physical measurements of the compound m-TAZDCz-nH are listed below:

1H-NMR (400 MHz, CDCl3, 5): 8.06 (d, J=8 Hz, 2H), 7.87 (d, 1H), 7.52-7.20 (m, 11H), 6.93 (d, J=8 Hz, 1H), 6.68 (d, J=8 Hz, 2H), 3.93 (t, J1=4 Hz, J2=12 Hz, 2H), 1.42 (m, 2H), 1.03-0.98 (m, 6H), 0.73-0.70 (t, J1=8 Hz, J2=12 Hz).

13C-NMR (100 MHz, CDCl3, δ): 155.73, 154.73, 140.55, 129.69, 128.98, 128.58, 128.09, 127.04, 127.06, 126.80, 125.58, 123.29, 120.03, 119.82, 114.11, 109.73, 68.91, 31.06, 28.53, 25.16, 22.20, 13.75.

Properties of Compounds CzDBS and CzDBSO

The glass transition temperature (Tg), melting point (Tm), decomposition temperature (Td), band gap (Eₛ), triplet energy gap (E_{T}), and HOMO (highest occupied molecular orbital) and LUMO (lowest unoccupied molecular orbital) energy gap of compounds m-TAZCz, m-TAZtCz, m-TAZDCz, and m-TAZDtCz were measured and are shown in Table 2.

TABLE 2

| | m-TAZCz | m-TAZtCz | m-TAZDCz | m-TAZDtCz |
|---|---|---|---|---|
| T_{g} | — | 120.6° C. | 159° C. | — |
| Tₘ | 312.4° C. | 288.7° C. | 301.06° C. | — |
| T_{d} (5%) | 358° C. | 367° C. | 432° C. | 421° C. |
| LUMO | 2.31 | 1.92 | 2.03 | 2.13 |
| HOMO | 5.99 | 5.54 | 5.6 | 5.68 |
| E_{T} | 3.0 | 2.99 | 3.01 | 2.97 |
| Es | 3.68 | 3.62 | 3.57 | 3.54 |

As shown in Table 2, since the compounds m-TAZCz, m-TAZtCz, and m-TAZDCz have decomposition temperatures (Td) of more than 350° C. (m-TAZDCz has a decomposition temperatures (Td) of more than 430° C. especially), and glass transition temperatures Tg) of more than 120° C., the compounds of the disclosure exhibits excellent thermal stability. The compounds also have suitable LUMO and HOMO energy gaps, thereby substantially matching a normal electron transfer layer.

Organic Electroluminescent Device

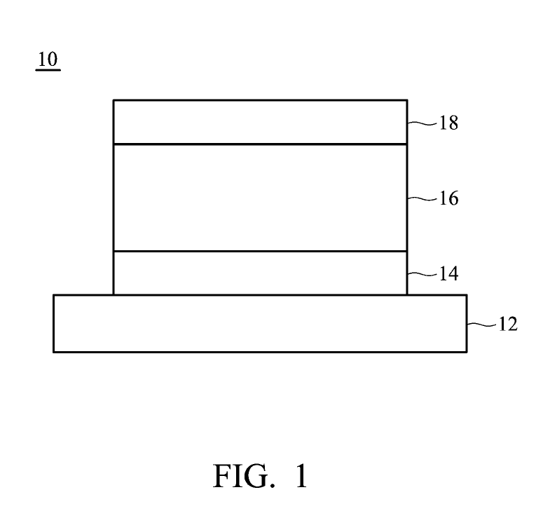

FIG. 1 shows an embodiment of an organic electroluminescent device 10. The electroluminescent device 100 includes a substrate 12, a bottom electrode 14, an electroluminescent element 16, and a top electrode 18, as shown in FIG. 1. The organic electroluminescent device can be top-emission, bottom-emission, or dual-emission devices.

The substrate 12 can be a glass plastic, or semiconductor substrate. Suitable materials for the bottom and top electrodes can be Ca, Ag, Mg, Al, Li, In, Au, Ni, W, Pt, Cu, indium tin oxide (ITO), indium zinc oxide (IZO), aluminum zinc oxide (AZO), or zinc oxide (ZnO), formed by sputtering, electron beam evaporation, thermal evaporation, or chemical vapor deposition. Further, al least one of the bottom and top electrodes 14 and 18 is transparent.

The electroluminescent element 16 at least includes an emission layer, and can further include a hole injection layer, a hole transport layer, an electron transport layer, and an electron injection layer. In an embodiment of the invention, at least one layer of the electroluminescent element 16 includes the aforementioned organic compound.

According to an embodiment of the invention, the organic electroluminescent device can be a phosphorescent organic electroluminescent device, and the phosphorescent organic electroluminescent device can include an emission layer including a host material and a phosphorescent dopant, wherein the host material includes the aforementioned organic compounds.

In order to clearly disclose the organic electroluminescent devices of the invention, the following examples (using m-TAZCz (prepared from Example 1) as host materials and blue or green phosphorescent dopant) are intended to illustrate the disclosure more fully without limiting their scope, since numerous modifications and variations will be apparent to those skilled in this art.

### Example 6

### Green Organic Electroluminescent Device

A glass substrate with an indium tin oxide (ITO) film of 100 nm was provided and then washed with a cleaning agent, acetone, and isopropanol with ultrasonic agitation. After drying with nitrogen flow, the ITO film was subjected to a UV/ozone treatment. Next, TAPC (1,1-bis(di-4-tolylaminophenyl)cyclohexane, with a thickness of 40 nm), m-TAZCz doped with Irppy3((tris(2-phenylpyridine)iridium) (the ratio between m-TAZCz and Irppy3 was 100:15, with a thickness of 30 nm), BPhen (4,7-diphenyl-1,10-phenanthroline, with a thickness of 30 nm), LiF (with a thickness of 0.5 nm), and Al (with a thickness of 110 nm) were subsequently formed on the ITO film at 10-6 Pa, obtaining the electroluminescent device (1). The materials and layers formed therefrom are described in the following.

ITO (100 nm)/TAPC (40 nm)/Irppy3 (15%): m-TAZCz (30 nm)/BPhen (30 nm)/LiF (0.5 nm)/Al (110 nm)

The optical property of the electroluminescent device (1), as described in Example 6, was measured by a PR650 (purchased from Photo Research Inc.) and a Minolta TS110. The results are shown below:

Optimal efficiency: 31.9 cd/A, and 22.7 lm/W;

Emissive efficiency: 31.8 cd/A, and 22.2 lm/W (@1000 cd/m2);

CIE coordinations: (0.30, 0.63)

### Example 7

### Blue Organic Electroluminescent Device

A glass substrate with an indium tin oxide (ITO) film of 100 nm was provided and then washed with a cleaning agent, acetone, and isopropanol with ultrasonic agitation. After drying with nitrogen flow, the ITO film was subjected to a UV/ozone treatment. Next, NPB (N,N′-di(naphthalene-1-yl)-N,N′-diphenyl-benzidine, with a thickness of 50 nm), TAPC (1,1-bis(di-4-tolylaminophenyl)cyclohexane, with a thickness of 15 nm), m-TAZCz doped with Firpic (Iridium-bis(4,6-difluorophenyl-pyridinato-N,C2)-picolinate) (the ratio between m-TAZCz and Irppy3 was 100:11, with a thickness of 40 nm), m-TPhOXD

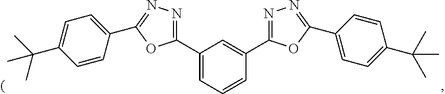 [SMILES: CC(C)(C)c1ccc(-c2nnc(-c3cccc(-c4nnc(-c5ccc(C(C)(C)C)cc5)o4)c3)o2)cc1]

with a thickness of 12.5 nm), BPhen(4,7-diphenyl-1,10-phenanthroline), with a thickness of 12.5 nm), LiF (with a thickness of 1 nm), and Al (with a thickness of 100 nm) were subsequently formed on the ITO film at 10-6 Pa, obtaining the electroluminescent device (2). The materials and layers formed therefrom are described in the following.

NPB (50 nm)/TAPC (15 nm)/m-TAZCz:Firpic 11% (40 nm)/m-TPhOXD (12.5 nm)/BPhen (12.5 nm)/LiF (1 nm)/Al (100 nm)

The optical property of the electroluminescent device (2), as described in Example 6, was measured by a PR650 (purchased from Photo Research Inc.) and a Minolta TS110. The results are shown below:

Optimal efficiency: 21.43 cd/A, 11.99 lm/W;

Emissive efficiency: 20 cd/A, 7.2 lm/W (@1000 cd/m2);

CIE coordinations: (0.16, 0.35)

The organic electroluminescent device employing the organic compounds of Formula (I) can have an emissive efficiency of 7 lm/W for emitting blue light (or 22 lm/w for emitting green light) at a brightness of 100 Cd/m2. Accordingly, the organic compounds of Formula (I) of the invention have a high triplet energy (tEg) gap and are apt to transmit energy to a guest emitter. The organic compounds of Formula (I) of the disclosure have a trizole moiety bound to the benzene at the meta-position relative to a carbazole moiety resulting in a shorter conjugated system. Therefore, the organic compounds of Formula (I) of the invention are suitable as host material of blue or green phosphorescent organic electroluminescent devices, thereby increasing efficiency thereof.

While the disclosure has been described by way of example and in terms of the preferred embodiments, it is to be understood that the disclosure is not limited to the disclosed embodiments. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. An organic compound having a Formula (I), of: 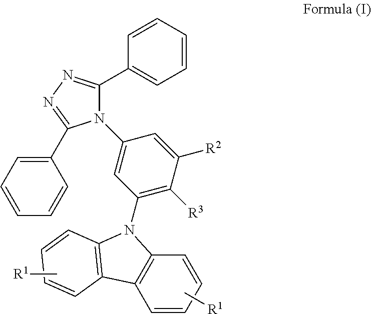
wherein R¹ are independent a hydrogen, or C₁₋₈ alkyl; and R², and R³ are independent a hydrogen, hydroxy, C₁₋₈ alkyl, C₁₋₈ alkoxy, C₅₋₁₀ aryl, or C₂₋₈ heteroaryl.

2. The organic compound as claimed in claim 1, wherein R¹, R², and are each independently a methyl group, ethyl group, propyl group, isopropyl group, butyl group, tert-butyl group, pentyl group, or hexyl group.

3. The organic compound as claimed in claim 1, wherein R², and R³ are each independently a methoxy group, ethoxy group, propoxy group, isopropoxy group, butoxy group, isobutoxy group, phenyl, biphenyl, pyridyl, furyl, naphthyl, anthryl, phenanthrenyl, imidazolyl, pyrimidinyl, quinolinyl, indolyl, or thiazolyl.

4. The organic compound as claimed in claim 1, wherein the organic compound has a Formula (II), of: 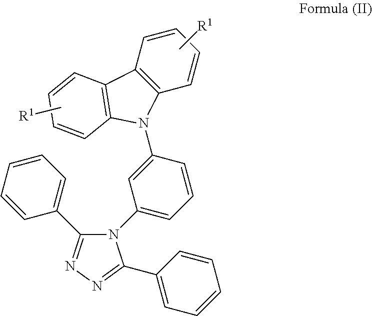
wherein R¹ are independent a hydrogen, or C₁₋₈ alkyl.

5. The organic compound as claimed in claim 4, wherein the organic compound comprises

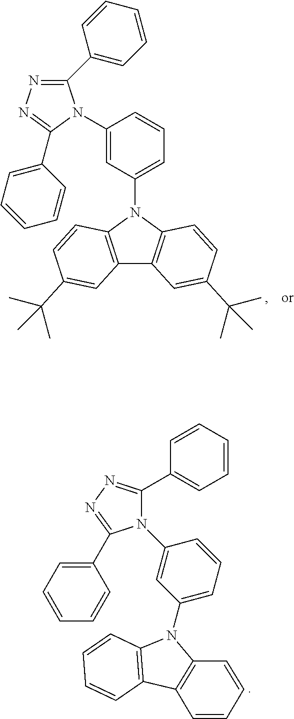 [SMILES: CC(C)(C)c1ccc2c(c1)c1cc(C(C)(C)C)ccc1n2-c1cccc(-n2c(-c3ccccc3)nnc2-c2ccccc2)c1 ; c1ccc(-c2nnc(-c3ccccc3)n2-c2cccc(-n3c4ccccc4c4ccccc43)c2)cc1]

6. The organic compound as claimed in claim 1, wherein the organic compound has a Formula (III), of: 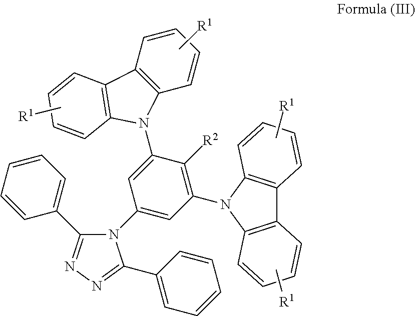
wherein R¹ are independent a hydrogen, or C₁₋₈ alkyl; and
R² are independent a hydrogen, hydroxy, C₁₋₈ alkyl, or C₁₋₈ alkoxy.

7. The organic compound as claimed in claim 6, wherein the organic compound comprises

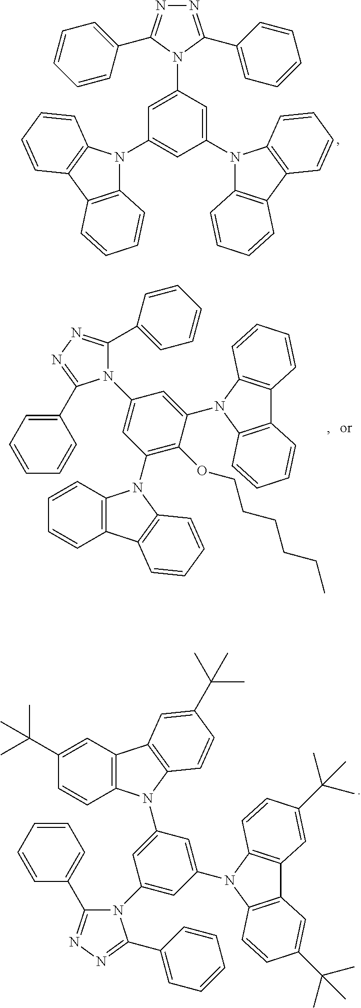

8. An organic electroluminescence device, comprising:
a pair of electrodes; and
an electroluminescent element disposed between the pair of electrodes, wherein the electroluminescent element comprises the organic compound as claimed in claim 1.

9. An organic electroluminescence device, comprising:
a pair of electrodes; and
an electroluminescent element disposed between the pair of electrodes, wherein the electroluminescent element comprises an emission layer comprising a host material and a phosphorescent dopant, and the host material comprises the organic compound as claimed in claim 1.

10. The organic electroluminescent device as claimed in claim 9, wherein the emission layer emits blue or green light under a bias voltage.
